(19) United States
(12) Patent Application Publication
(10) Pub. No.: US 2022/0296510 A1
(43) Pub. Date: Sep. 22, 2022

(54) SELF-SIZING DEVICE FOR DELIVERING A FORMULATION TO A LUMEN WALL

(71) Applicant: Rani Therapeutics, LLC, San Jose, CA (US)

(72) Inventor: Mir IMRAN, Los Altos Hills, CA (US)

(73) Assignee: Rani Therapeutics, LLC, San Jose, CA (US)

(21) Appl. No.: 17/709,245

(22) Filed: Mar. 30, 2022

Related U.S. Application Data
(63) Continuation of application No. PCT/US2020/053619, filed on Sep. 30, 2020.
(60) Provisional application No. 62/909,206, filed on Oct. 1, 2019.

(51) Int. Cl. A61K 9/00

(52) U.S. Cl. CPC A61K 9/0065; A61K 9/0097

(57) ABSTRACT

A system is provided for delivering a therapeutic formulation into a lumen. The system includes a self-sizing device comprising an expandable component that includes at least one non-compliant section structured to resist deformation in an expanded configuration, the expandable component further including at least one hinge structured to allow deformation in an expanded configuration, the expandable component structured to bend about the hinge upon expansion to adapt to an inner circumference of a lumen independent of the inner circumference of the lumen within a selected range. The system includes the therapeutic formulation and a delivery mechanism. The self-sizing device is structured to, upon expansion of the expandable component, cause the delivery mechanism to apply a force to the therapeutic formulation, the force designed to expel the therapeutic formulation from the self-sizing device. The self-sizing device may be disposed within a capsule.

### CROSS-REFERENCE TO RELATED APPLICATIONS

This application claims priority to, and the benefit of, U.S. Provisional Patent Application No. 62/909,206 titled INFLATABLE DEVICES, SYSTEMS AND METHODS FOR DELIVERING THERAPEUTIC COMPOUNDS INTO WALL OF THE GASTRO INTESTINAL TRACT, filed on Oct. 1, 2019, which is incorporated herein by reference in its entirety for all purposes.

### BACKGROUND

It may be desirable to deliver a material or formulation (e.g., a fluid, slurry, powder, or solid) into a lumen, and it may further be desirable to deliver the material at or into an inner wall of the lumen. In many applications, an inner circumference of a lumen may be unpredictable and/or may be variable, or inner circumferences of multiple lumens collectively may be unpredictable and/or may be variable, such that selecting a device effective to deliver the material at or into the inner wall of any given lumen may present a challenge.

An example of delivery into a lumen of an animalia body is delivery of a therapeutic formulation into a lumen of the gastrointestinal tract. Delivery of therapeutic formulations by way of a pill that dissolves in the gastrointestinal tract is not effective for many therapeutic formulations due to a failure of active agents in the therapeutic formulation to pass into a wall of the gastrointestinal tract and thus reach vasculature, or due to destruction of the active agents within the gastrointestinal tract by gastric juices or other fluids or matter in the gastrointestinal tract, among other reasons. To address these concerns, some oral delivery devices have been developed that are designed to activate within the gastrointestinal tract to deliver therapeutic formulations into a wall of the gastrointestinal tract to reach vasculature. Obstacles faced by such devices include unpredictability and variability between the several lumens of the gastrointestinal tract, unpredictability and variability within a single lumen of the gastrointestinal tract, unpredictability and variability between lumens of different species, and unpredictability and variability between lumens in different subjects of a same species. These obstacles present a challenge for identifying a device that will be effective to deliver therapeutic formulations at a target delivery site within the gastrointestinal tract and accommodate the various lumen inner circumferences that may be encountered at that target delivery site.

Similar challenges present themselves with respect to other lumens within the animalia body, and with respect to other types of lumens in general.

### SUMMARY

Embodiments of the present disclosure provide devices, systems, and methods for delivering a formulation to a lumen wall independent of an inner circumference of the lumen.

In an aspect, a self-sizing device for delivering a therapeutic formulation includes a capsule sized and structured to be orally ingested, and an expandable component disposed within the capsule. The expandable component includes at least one non-compliant section structured to resist deformation in an expanded configuration and at least one hinge structured to allow deformation in an expanded configuration. The expandable component is structured to expand within a lumen and bend about the hinge to adapt to an inner circumference of the lumen, independent of the inner circumference within a selected range.

In an embodiment, the selected range is about 50 mm to about 150 mm.

In an embodiment, the selected range is about 5 mm to about 500 mm.

In an embodiment, while in the capsule, the expandable component is in a folded and/or rolled arrangement and is structured to unfold and/or unroll when released from the capsule to expand to a maximum circumference in the absence of a constraining force and to less than a maximum circumference in the presence of a constraining force.

In an embodiment, the hinge has a width or a circumference smaller than a respective width or circumference of the non-compliant section.

In an embodiment, the lumen is a lumen of the small intestine.

In an embodiment, the expandable component comprises at least two non-compliant sections.

In an embodiment, the expandable component comprises at least two hinges.

In an embodiment, the self-sizing device further includes the therapeutic formulation and a piston, and the self-sizing device is structured to, upon expansion of the expandable component, cause the piston to apply a force to the therapeutic formulation, the force designed to expel the therapeutic formulation from the device.

In an aspect, a system for delivering a formulation includes a self-sizing device, a formulation, and a delivery mechanism. The self-sizing device includes an expandable component that includes at least one non-compliant section structured to resist deformation in an expanded configuration, and at least one hinge structured to allow deformation in an expanded configuration. The expandable component is structured to bend about the hinge upon expansion to adapt to an inner circumference of a lumen independent of the inner circumference of the lumen within a selected range. The self-sizing device is structured to, upon expansion of the expandable component, cause the delivery mechanism to apply to the formulation a force designed to expel the formulation from the self-sizing device.

In an embodiment, the system further includes a capsule in which the expandable component is disposed. While in the capsule, the expandable component is in a folded and/or rolled arrangement and is structured to unfold and/or unroll when released from the capsule.

In an embodiment, the expandable component is further structured to, after being released from the capsule, expand to a maximum circumference in the absence of a constraining force and expand to less than a maximum circumference in the presence of a constraining force.

In an embodiment, the self-sizing device is structured to be disposed in a gastrointestinal tract, and the selected range is about 50 mm to about 150 mm.

In an embodiment, the hinge has a width or a circumference smaller than a respective width or circumference of the non-compliant section.

In an embodiment, the lumen is a lumen of the small intestine.

In an embodiment, the expandable component comprises at least two non-compliant sections.

In an embodiment, the expandable component comprises at least two hinges.

In an embodiment, the delivery mechanism comprises a piston, and the self-sizing device is structured such that upon expansion of the expandable component the piston applies the force to the formulation.

In an aspect, a method for delivering a therapeutic formulation includes providing a self-sizing device comprising the therapeutic formulation, the self-sizing device further comprising an expandable component that includes at least one non-compliant section structured to resist deformation in an expanded configuration and at least one hinge structured to allow deformation in an expanded configuration. The expandable component is structured to bend about the hinge upon expansion to adapt to an inner circumference of a lumen independent of the inner circumference of the lumen within a selected range. The method further includes providing instructions for disposing the self-sizing device within a body to deliver the therapeutic formulation into the lumen.

In an embodiment, the instructions include instructions for swallowing the self-sizing device.

In an embodiment, the instructions include instructions for manually inserting the self-sizing device into the lumen.

### BRIEF DESCRIPTION OF THE DRAWINGS

FIG. 1, FIG. 2, FIG. 3, and FIG. 4 each illustrate an example of an embodiment of an expandable component with at least two non-compliant sections and at least one hinge.

FIG. 5A, FIG. 5B, FIG. 5C, FIG. 5D, FIG. 5E, and FIG. 5F each illustrate an example of an embodiment of an expandable component with at least two non-compliant sections and at least one hinge.

FIG. 6 illustrates an example of an embodiment of an expandable component with at least two non-compliant sections and at least two hinges.

FIG. 7A and FIG. 7B illustrate an example of an embodiment of an expandable component with at least one non-compliant section and at least two hinges.

FIG. 8A and FIG. 8B illustrate an example of an embodiment of an expandable component with at least two non-compliant sections and at least two hinges.

FIG. 9A and FIG. 9B each illustrate an example of an embodiment of a capsule structure.

FIG. 10A illustrates an example of an embodiment of an expandable component prior to being folded and/or rolled, and an example of an embodiment of a capsule.

FIG. 10B illustrates the expandable component of FIG. 10A in an embodiment of a folded and/or rolled arrangement prior to disposing the expandable component in the capsule.

FIG. 10C illustrates the expandable component of FIG. 10A in the folded and/or rolled arrangement of FIG. 10B and disposed within the capsule.

FIG. 11A, FIG. 11B, and FIG. 11C illustrate an example of an embodiment of a swallowable device including an expandable component within a degradable capsule, as the device traverses a lumen.

FIG. 11D, FIG. 11E, and FIG. 11F illustrate a progression of the expandable component of FIG. 11C in a rotated view as the expandable component expands within the lumen.

FIG. 12 illustrates an example of an embodiment of a self-sizing device including an expandable component in a fully-extended state within a lumen.

FIG. 13A illustrates an example of an embodiment of a self-sizing device including an expandable component with one non-compliant section and no hinge.

FIG. 13B illustrates an example of an embodiment of a self-sizing device including an expandable component with two non-compliant sections and one hinge.

### DETAILED DESCRIPTION

The present disclosure relates to self-sizing devices for the delivery of formulations into a lumen. Before discussing details of the self-sizing devices of the present disclosure, a few conventions are provided for the convenience of the reader.

Various abbreviations may be used herein for standard units, such as deciliter (dl), milliliter (ml), microliter (μl), international unit (IU), centimeter (cm), millimeter (mm), nanometer (nm), inch (in), kilogram (kg), gram (gm), milligram (mg), microgram (μg), millimole (mM), degrees Celsius (° C.), degrees Fahrenheit (° F.), millitorr (mTorr), hour (hr), or minute (min).

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural referents unless the context clearly dictates otherwise. Reference to an object in the singular is not intended to mean “one and only one” unless explicitly so stated, but rather “one or more.”

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

The term “component” refers herein to one item of a set of one or more items that together make up a device, formulation or system under discussion. A component may be in a solid, powder, gel, plasma, fluid, gas, or other constitution. For example, a device may include multiple solid components which are assembled together to structure the device and may further include a fluid component that is disposed in the device. For another example, a formulation may include two or more powdered and/or fluid components which are mixed together to make the formulation.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, pH, species, diet, health, gender, age, ancestry, disease, or tissue damage); it is to be understood that actual tolerances and environmental conditions before and/or after delivery can affect characteristics so that different components, devices, formulations, or systems with a same design can have different actual values with respect to those characteristics. Design encompasses also variations or modifications before or after manufacture.

The term “manufacture” or a grammatical variation thereof (e.g., “manufacturing” or “manufactured”) as related to a component, device, formulation, or system refers herein to making or assembling the component, device, formulation, or system. Manufacture may be wholly or in part by hand and/or wholly or in part in an automated fashion.

The term “structured” or a grammatical variation thereof (e.g., “structure” or “structuring”) refers herein to a component, device, formulation, or system that is manufactured according to a concept or design or variations thereof or modifications thereto (whether such variations or modifications occur before, during, or after manufacture) whether or not such concept or design is captured in a writing.

The term “body” refers herein to an animalia body.

The term “subject” refers herein to a body into which an embodiment of the present disclosure is, or is intended to be, delivered. For example, with respect to humans, a subject may be a patient under treatment of a health care professional.

The term “fluid” refers herein to a liquid or gas, and encompasses moisture and humidity. The term “fluidic environment” refers herein to an environment in which one or more fluids are present.

The term “biological matter” refers herein to blood, tissue, fluid, enzymes, interstitial fluid, and other secretions of a body. The term “digestive matter” refers herein to biological matter along the gastrointestinal (GI) tract in an animalia body, and other matter (e.g., food in an undigested or a digested state such as chyme) traversing the gastrointestinal tract.

The term “ingest” or a grammatical variation thereof (e.g., “ingesting” or “ingested”) refers herein to taking into the stomach, whether by swallowing or by other means of depositing into the stomach (e.g., by depositing into the stomach by endoscope or depositing into the stomach via a port).

The term “lumen” refers herein to the inside space of a tubular structure. Examples of lumens in a body include arteries, veins, and tubular cavities within organs.

The term “lumen wall” refers to a wall of a lumen, where the wall includes all layers from an inner perimeter to an outer perimeter of the lumen, such as, with respect to lumens in a body, the mucosa, submucosa, muscularis, serosa, and an outer wall of the lumen, with the constituent blood vessels and tissues.

The term “gastrointestinal tract” or “GI tract” refers herein to the intake/expulsion system of a body including, for example, the mouth, pharynx, esophagus, stomach, pylorus, small intestine, cecum, large intestine, colon, rectum, anus, and valves or sphincters therebetween.

The term “GI lumen” refers generally to any lumen of the GI tract (e.g., a lumen of the esophagus, stomach, small intestine, large intestine, or colon) and the term “GI lumen wall” refers to a lumen wall of a GI lumen.

The term “formulation” refers herein to a preparation including one or more components. A formulation may be constituted of a fluid, a slurry, a powder, or a solid (e.g., in a condensed or a consolidated form such as a tablet or microtablet). Each formulation can include one or more components, and a device or system can include one or more formulations.

The term “therapeutic formulation” refers herein to a formulation intended for a therapeutic, diagnostic, or other biological purpose. A component of a therapeutic formulation can be, for example, a pharmacologically active agent, a DNA or SiRNA transcript, a cell, a cytotoxic agent, a vaccine or other prophylactic agent, a nutraceutical agent, a vasodilator, a vasoconstrictor, a delivery enhancing agent, a delay agent, an excipient, a diagnostic agent, or a substance for cosmetic enhancement.

A pharmacologically active agent can be, for example, an antibiotic, a nonsteroidal anti-inflammatory drug (NSAID), an angiogenesis inhibitor, a neuroprotective agent, a chemotherapeutic agent, a peptide, a protein, an immunoglobulin (e.g., a TNF-alpha antibody), an interleukin in the IL-17 family of interleukins, an anti-eosinophil antibody, another antibody, a nanobody, a large molecule, a small molecule, or a hormone, or a biologically active variant or derivative of any of the foregoing.

A cell can be, for example, a stem cell, a red blood cell, a white blood cell, a neuron, or other viable cell. Cells can be produced by or from living organisms or contain components of living organisms. A cell can be allogeneic or autologous.

A vaccine can be, for example, against an influenza, a coronavirus, meningitis, human papillomavirus (HPV), or chicken pox. A vaccine can correspond to an attenuated virus.

A nutraceutical agent can be, for example, vitamin A, thiamin, niacin, riboflavin, vitamin B-6, vitamin B-12, another B-vitamin, vitamin C (ascorbic acid), vitamin D, vitamin E, folic acid, phosphorous, iron, calcium, or magnesium.

A vasodilator can be, for example, 1-arginine, sildenafil, a nitrate (e.g., nitroglycerin), or epinephrine.

A vasoconstrictor can be, for example, a stimulant, an amphetamine, an antihistamine, epinephrine, or cocaine.

A delivery enhancement agent can be, for example, a permeation enhancer, an enzyme blocker, a peptide that permeates through mucosa, an antiviral drug such as a protease inhibitor, a disintegrant, a superdisintegrant, a pH modifier, a surfactant, a bile salt, a fatty acid, a chelating agent, or a chitosan. A delivery enhancing agent can, for example, serve as a delivery medium for delivery of a component of a therapeutic formulation, or serve to improve absorption of a component of a therapeutic formulation into the body. A delivery enhancing agent can prime an epithelium of the intestine (e.g., fluidize an outer layer of cells) to improve absorption and/or bioavailability of one or more other components included in the delivery device.

A delay agent can be, for example, poly(lactic acid) (PLA), poly(glycolic acid) (PGA), polyethylene glycol (PEG), poly(ethylene oxide) (PEO), poly (l-lactic acid) (PLLA), poly(D-lactic acid) (PDLA), another polymer, or a hydrogel. A delay agent can be included with (e.g., mixed with, or providing a structure around) one or more other component(s) in a therapeutic formulation to slow a release rate of the other component(s) from the therapeutic formulation.

An excipient can be, for example, a binder, a disintegrant, a superdisintegrant, a buffering agent, an anti-oxidant, or a preservative. Excipients can provide a medium for a component of a therapeutic formulation (e.g., for assisting in manufacture), or to preserve integrity of a component of a therapeutic formulation (e.g., during manufacture, during storage, or after ingestion prior to dispersion within the body).

A diagnostic agent can be, for example, a sensing agent, a contrast agent, a radionuclide, a fluorescent substance, a luminescent substance, a radiopaque substance, or a magnetic substance.

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking. The term “non-degradable” refers to an expectation that degradation will be minimal, or within a certain acceptable design percentage, for at least an expected duration in an expected environment.

The terms “substantially” and “about” are used herein to describe and account for small variations. For example, when used in conjunction with a numerical value, the terms can refer to a variation in the value of less than or equal to ±10%, such as less than or equal to +5%, less than or equal to ±4%, less than or equal to ±3%, less than or equal to +2%, less than or equal to ±1%, less than or equal to +0.5%, less than or equal to +0.1%, or less than or equal to +0.05%.

As used herein, a range of numbers includes any number within the range, or any sub-range if the minimum and maximum numbers in the sub-range fall within the range. Thus, for example, “<9” can refer to any number less than nine, or any sub-range of numbers where the minimum of the sub-range is greater than or equal to zero and the maximum of the sub-range is less than nine. Ratios may also be presented herein in a range format. For example, a ratio in the range of about 1 to about 200 should be understood to include the explicitly recited limits of about 1 and about 200, and also to include individual ratios such as about 2, about 35, and about 74, and sub-ranges such as about 10 to about 50, about 20 to about 100, and so forth.

The discussion now continues with respect to self-sizing devices. Embodiments of the present description provide devices, systems, and methods of manufacture for self-sizing devices.

In an embodiment, a self-sizing device is a device designed to deliver therapeutic formulations at or into a lumen wall. In an embodiment, a self-sizing device is an ingestible device designed to deliver therapeutic formulations at or into a GI lumen wall; such a device may be delivered within a capsule.

The self-sizing device includes a component structured to be expanded, referred to hereinafter for convenience as an “expandable component”. The self-sizing device includes at least one expansion module that when triggered causes the expandable component to expand from a first unexpanded configuration to a second expanded configuration.

The expandable component includes multiple sections. In the expanded configuration of the expandable component, at least one of the sections is non-compliant and at least one of the sections is compliant. Compliant refers to a state in which the section may be readily deformed, and non-compliant refers to a state in which the section resists deformation.

Absent a constraint, each section will expand to a fully-extended state and the expandable component will reach its maximum dimensions, which may depend, for example, on materials used to form the expandable component and/or a capacity of the expansion module that is implemented (e.g., for expansion by inflation, maximum dimensions may be influenced by a limitation on an inflation force available from the expansion module, or by a stretch factor of a material or materials of the expandable component).

Each compliant section operates in similar fashion to a hinge and thus will be referred to for convenience as a hinge. In a fully-extended state of the expandable component (where each section is fully extended), each hinge has at least one design dimension (width, length, and/or circumference) that is substantially smaller than a corresponding design dimension of each non-compliant section.

As the expandable component expands, it will achieve a shape that reflects an equilibrium between a force exerted by the expandable component on an interior of the lumen and a force of the lumen against the expandable component. Said another way, the expandable component may not fully extend and will tend to be bent about the hinge if constrained by the lumen. This bending is due to the smaller dimension(s) of the hinge as compared to the corresponding dimension(s) of the non-compliant section(s), which in the expanded configuration of the expandable component leads to lower rigidity of the hinge as compared to rigidity of the non-compliant section(s).

In this way, the expandable component expands until the non-compliant sections (which may hereinafter be referred to as NCS) are pressed against the lumen inner wall to maintain the expandable component in a position suitable for delivering a formulation to the lumen wall, for a time at least sufficient to accomplish such delivery. The expandable component may then be deflated either automatically or manually.

The expandable component may be removed after deflation, may be left to degrade in situ, or may be allowed to pass out of the lumen. For example, when used in an application within a GI lumen, the expandable component may be allowed to pass through the GI tract after deflation. The expandable component can be structured from degradable materials such that the expandable component degrades after a designed period of time after exposure to an environment at a target site.

The self-sizing device is particularly suited to avoid delivery being affected by matter present in a lumen. For example, the self-sizing device can have a capability by rapid expansion of the expandable component to push matter out of the way and/or to compress matter against the lumen wall, expanding to the extent appropriate for a desired delivery rate for the delivery technique and the formulation to be delivered without further expansion. For a self-sizing device structured for delivery into a GI lumen, the self-sizing device can accomplish such delivery whether the subject is in a fed or fasted state (meaning, that delivery is accomplished whether there is digestive matter in the GI lumen or not).

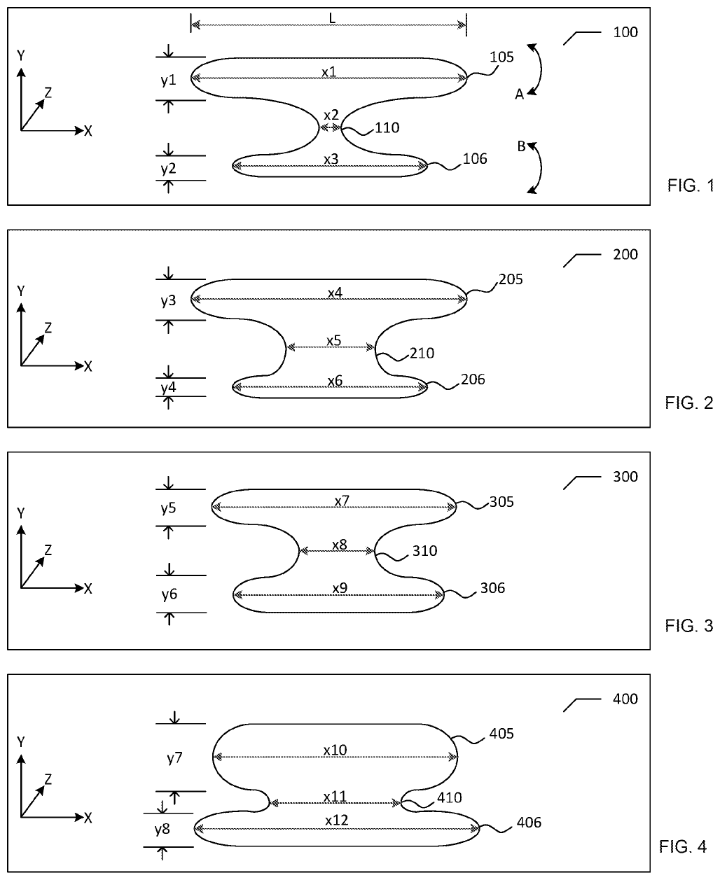

FIGS. 1-4 illustrate examples of embodiments of expandable components in fully-extended states in which the expandable components are extended without constraint to maximum dimensions (which may depend, for example, on material(s) used for the expandable components and a capability of an expansion module, among other considerations). An arbitrarily assigned x-y-z frame of reference is indicated for convenience of discussion, and the expandable components of FIGS. 1-4 are shown in an x-y reference plane. Each expandable component includes two NCSs and one hinge in the x-y plane shown.

Referring to FIG. 1, an expandable component 100 includes an NCS 105 and an NCS 106 separated by a hinge 110. In this view of this embodiment as illustrated in FIG. 1A, a dimension x1 of NCS 105 is greater than a dimension x3 of NCS 106, and both x1 and x3 are greater than a dimension x2 of hinge 110. Said another way, x1/x3>1, x1/x2>1, and x3/x2>1. Further in this view of this embodiment, a dimension y1 of NCS 105 is greater than a dimension y2 of NCS 106. In this view, then, NCS 105 is taller and longer than NCS 106.

Often, the elongated shape of an expandable component (e.g., expandable component 100) will result in the expandable component having a long dimension (e.g., length L) aligned with a central axis of the lumen in which the expandable component is positioned.

In the embodiment illustrated in FIG. 1, because hinge 110 is much narrower than NCS 105 and NCS 106 (x2<<x1 and x2<<x3, respectively), NCS 105 and NCS 106 may bend towards or away from each other in the x-y plane around hinge 110 as indicated by the arrows labeled A and B, which can allow expandable component 100 to accommodate an uneven lumen surface and/or inconsistent lumen diameter.

Referring to FIG. 2, an expandable component 200 includes an NCS 205 and an NCS 206 separated by a hinge 210. Expandable component 200 is similar in this view to expandable component 100 as shown in FIG. 1. A dimension x4 of NCS 205 is greater than a dimension x6 of NCS 206, and both x4 and x6 are greater than a dimension x5 of hinge 210; and a dimension y3 of NCS 205 is greater than a dimension y4 of NCS 206. However, x5 as shown in FIG. 2 is approximately three times greater than x2 as shown in FIG. 1; accordingly, NCS 205 and NCS 206 may have a capability to bend towards or away from each other in the x-y plane that is less than the capability of NCS 105 and NCS 106 to bend towards or away from each other in the x-y plane, while still maintaining some flexibility to allow expandable component 200 to accommodate an uneven lumen surface and/or inconsistent lumen diameter.

As seen by comparing FIG. 1 and FIG. 2, a dimension of a hinge (e.g., hinge 110 or hinge 210) can be designed to have a desired width. Width and other dimensions of an expandable component can be designed, for example, to be suitable for a particular application, to minimize an amount of material used to structure the expandable component, to reduce manufacturing costs, or to increase manufacturing speed.

Referring to FIG. 3, an expandable component 300 includes an NCS 305 and an NCS 306 separated by a hinge 310. A dimension x7 of NCS 305 is greater than a dimension x9 of NCS 306, and both x7 and x9 are greater than a dimension x8 of hinge 310. In this embodiment, a dimension y5 of NCS 305 is approximately equal to a dimension y6 of NCS 306, illustrating another variation of dimensions.

Referring to FIG. 4, an expandable component 400 includes an NCS 405 and an NCS 406 separated by a hinge 410. In this embodiment, a dimension x10 of NCS 405 is less than a dimension x12 of NCS 406, x10 and x12 are both somewhat greater than a dimension x11 of hinge 410, and a dimension y7 of NCS 405 is approximately two times greater than a dimension y8 of NCS 406, illustrating another variation of dimensions.

Other comparative dimensions of NCSs and hinges are within the scope of the present disclosure. For example, a dimension of a hinge in an x-y plane may be greater than a dimension of an NCS in that plane. Further, dimensions may vary in the z-direction within a particular section (NCS or hinge).

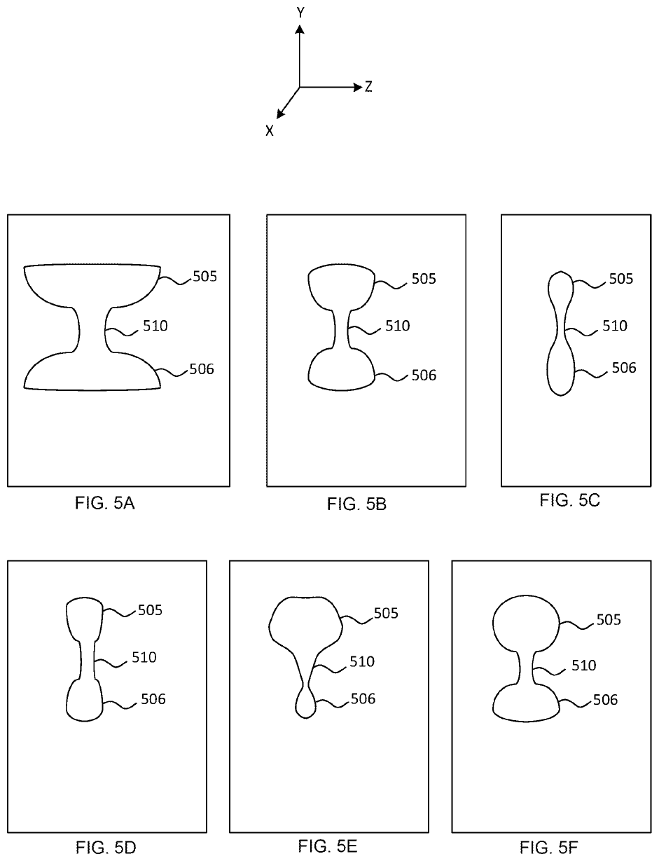

FIGS. 5A-5F illustrate examples of embodiments of expandable components, such as how one or more of expandable components 100, 200, 300, or 400 of FIGS. 1, 2, 3, 4 respectively might look as viewed when rotated (here, rotated into a y-z plane). For convenience with respect to FIGS. 5A-5F, non-compliant sections are referenced as NCS 505, 506 and compliant sections are referenced as hinge 510.

NCS 505 and NCS 506 may bend towards or away from each other in the y-z plane around hinge 510, which can allow the expandable component to accommodate a variety of lumen diameters and shapes.

As can be seen in FIGS. 5A-5F, a shape of an expandable component can vary in a rotated (e.g., side) view, as was the case for the unrotated (e.g., front) view. For example, one (or both) of NCS 505, 506 may have a surface that is substantially flat, somewhat rounded, quite rounded, or other curvature.

It will be apparent from the discussions above that each of the various sections (each NCS and each hinge) may be designed to have desired absolute dimensions as well as desired dimensions comparative to other sections. For example, a hinge may have a width in an x-y plane that is equal to or greater than a width of a particular NCS in the same x-y plane while having a width in a y-z plane that is substantially less than a width of that particular NCS in that y-z plane.

Viewing FIGS. 1-4 and FIGS. 5A-5F with respect to some examples of embodiments of expandable components, it will be apparent that there may be a broad variety of designs for expandable components having two non-compliant sections (NCS) and one compliant section (hinge) in accordance with the present disclosure. In various embodiments, expandable components in accordance with the present disclosure more generally have one or more NCS and one or more hinge.

An expandable component of a self-sizing device may be structured to have one or more sections in each of two or more planes. For example, expandable component 100 shown in a first x-y plane in FIG. 1 may be structured to have additional NCS and/or hinge sections in a second x-y plane such that, as viewed in a y-z plane, expandable component 100 forms a Y-shape, an X-shape, or other multi-pronged shape. A prong may include one or more NCS or no NCS, and/or one or more hinge or no hinge.

An expandable component may include a weakening feature to make it more compliant in a hinging area.

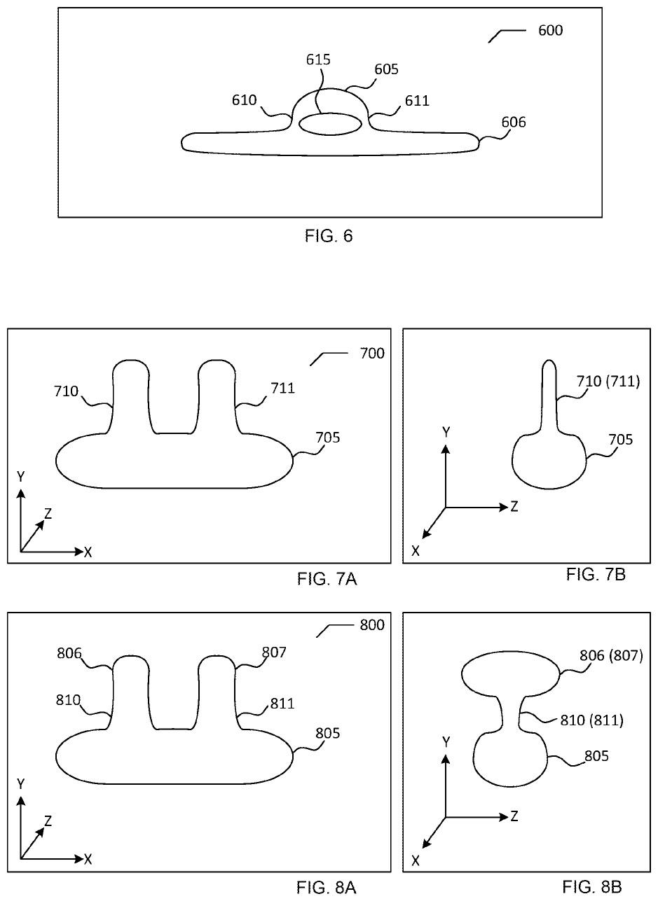

FIG. 6 illustrates an expandable component 600 having two NCSs in the view shown, an NCS 605 and an NCS 606. Expandable component 600 defines an opening 615 illustrated as an oval in this embodiment. Said in another way, expandable component 600 includes a weakening feature shown as opening 615. The omission of material in opening 615 leaves two hinges, a hinge 610 and a hinge 611. Hinges 610 and 611 as fully extended each have at least one design dimension (width, length, and/or circumference) that is substantially smaller than a corresponding design dimension of NCS 605 and NCS 606, which provides for an increase in compliance of hinges 610, 611 relative to NCS 605, 606. Expandable component 600 will tend to bend in this embodiment about hinges 610, 611. Hinges 610, 611 may have similar or different dimensions. When rotated to a different view, expandable component 600 may have a variety of shapes, including the shapes illustrated in FIGS. 5A-5F.

FIG. 7A and FIG. 7B illustrate an expandable component 700 having a single NCS 705 and two hinges, a hinge 710 and a hinge 711. Expandable component 700 is shown in an x-y plane in FIG. 7A, and shown in a y-z plane in FIG. 7B such that hinge 711 is hidden behind hinge 710. In this embodiment, hinges 710, 711 themselves operate to exert pressure against an inner surface of the lumen, while also providing compliance.

FIG. 8A and FIG. 8B illustrate an expandable component 800 having three NCSs, an NCS 805, an NCS 806, and an NCS 807, and two hinges, a hinge 810 and a hinge 811. Expandable component 800 is shown in an x-y plane in FIG. 8A, and shown in a y-z plane in FIG. 8B such that hinge 811 is hidden behind hinge 810 and NCS 807 is hidden behind NCS 806.

As can be seen from FIG. 7A and FIG. 8A, expandable component 700 and expandable component 800 look similar in the x-y planes shown. However, when rotated 90 degrees (e.g., as shown in FIG. 7B and FIG. 8B, respectively, shown in y-z planes), it is seen that NCS 806 and NCS 807 (FIG. 8B) expand to have a dimension in the z-direction that is substantially greater than the corresponding dimension of hinge 710 and 711 (FIG. 7B).

In an embodiment of a self-sizing device according to the present disclosure, an expandable component is disposed within a capsule.

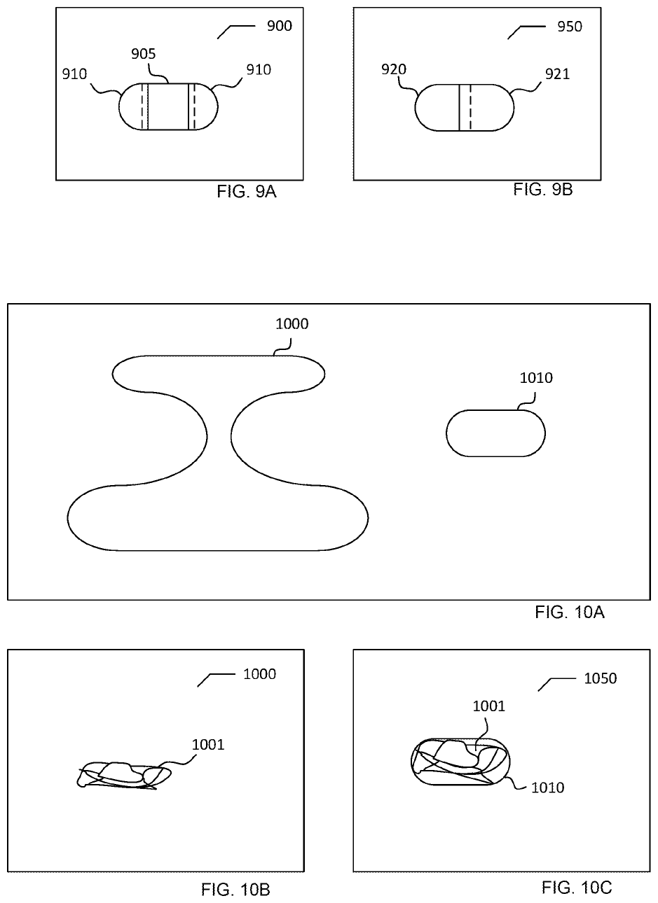

FIG. 9A and FIG. 9B illustrate examples of embodiments of capsules. In FIG. 9A, a capsule 900 includes a cylindrical body 905 and two end caps 910 which are fitted over or into body 905. At least one of the three components of capsule 900 (cylindrical body 905, a first end cap 910, and/or a second end cap 910) is degradable. End caps 910 may be adhered to or press fitted over or in body 905 to retain end caps 910 in position. In FIG. 9B, a capsule 950 includes a portion 920 and a portion 921 which are fitted together. At least one of portions 920, 921 is degradable. Portions 920, 921 may be adhered or press fitted together. Examples of capsule sizes include 000, 00, and 0 capsule sizes. Capsule shapes other than as illustrated in FIGS. 9A, 9B may also be used, such as spherical or ovoid shapes, or irregular shapes, or shapes that are symmetric about a first axis and asymmetric about a second axis perpendicular to the first axis (e.g., egg-shaped), or shapes with at least one flattened side.

In an embodiment, the self-sizing device includes a degradable coating over the expandable component and/or over the capsule (when a capsule is included).

In an embodiment, a coating is applied directly onto an expandable component to complete manufacture of a self-sizing device (e.g., instead of disposing the expandable component within a capsule). The coating may completely cover and seal the expandable component, or may cover a portion of the expandable component. For example, the coating may degrade under certain conditions to expose one or more degradable components of the self-sizing device. In turn, the one or more degradable components degrade after being exposed, such as to initiate expansion of the expandable component, or, in an embodiment in which a gas is used to inflate the expandable component, to release the gas and thus deflate the expandable component after delivery of a formulation.

The coating over the expandable device and/or the capsule may be, or may include, an opaque material (e.g., to keep the contents from view), a colored material (e.g., to provide color coding for identifying a formulation contained in the self-sizing device), and/or a material including a noticeable flavor or smell (e.g., undesirable flavor or smell to discourage interest, or desirable flavor or smell to encourage interest), each of which dissolves or degrades sufficiently to allow the self-sizing device to deliver a formulation at the target delivery site.

All, or portions of, the coating and/or the capsule may be designed to degrade after a certain length of time under the conditions expected at a target site, for example after a design period of time after placement, or in the presence of a particular chemical, or under certain values or ranges of pH, temperature, or pressure exerted against the capsule, or a combination of the foregoing. In an embodiment, the capsule is designed to break apart upon an external trigger, for example a trigger wirelessly sent which causes a mechanism within the capsule to rip or break the capsule.

As noted, an embodiment of a self-sizing device according to the present disclosure may be used to deliver a therapeutic formulation at or into a GI lumen wall via ingestion (e.g., by manual positioning of, or by swallowing, the self-sizing device). The self-sizing device may include a capsule. The self-sizing device may include a coating on the capsule and/or on the expandable component (e.g., a coating as described above), designed to degrade at a particular location within the GI tract, such as within the stomach or the intestine. In an embodiment, all or portions of the capsule and/or the coating are designed to degrade in the presence of a particular chemical, or after a certain length of time under the conditions expected at a target site within the GI tract (e.g., under certain values or ranges of pH, temperature, pressure exerted against the capsule, or a combination of the foregoing). In an embodiment, the coating includes a material to improve swallowability, such as guar gum or xanthan gum.

FIGS. 10A-10C illustrate an example of assembling a self-sizing device 1050. FIG. 10A illustrates an embodiment of an expandable component 1000 and an embodiment of a capsule 1010. Outer dimensions of expandable component 1000 may be several times larger than inner dimensions of capsule 1000. For example, a longest dimension of an embodiment of expandable component 1000 may be two to five times greater than an inner length of capsule 1010. Other ratios of outer dimensions of an expandable component to inner dimensions of a capsule are within the scope of the present disclosure. FIG. 10B illustrates expandable component 1000 folded and/or rolled into an arrangement 1001 that is smaller than inner dimensions of capsule 1010. As illustrated, arrangement 1001 is folded and/or rolled in a complex arrangement in this embodiment, origami style. FIG. 10C illustrates arrangement 1001 disposed into capsule 1010, and capsule 1010 assembled together (e.g., as in FIG. 9A in which end caps 910 are fitted over or into body 905, or as in FIG. 9B in which portions 920, 921 are fitted together). Device 1050 includes expandable component 1000 (in arrangement 1001) and capsule 1010, and may include other components not shown such as an expansion module, a delivery component, one or more coatings, and a deflation valve.

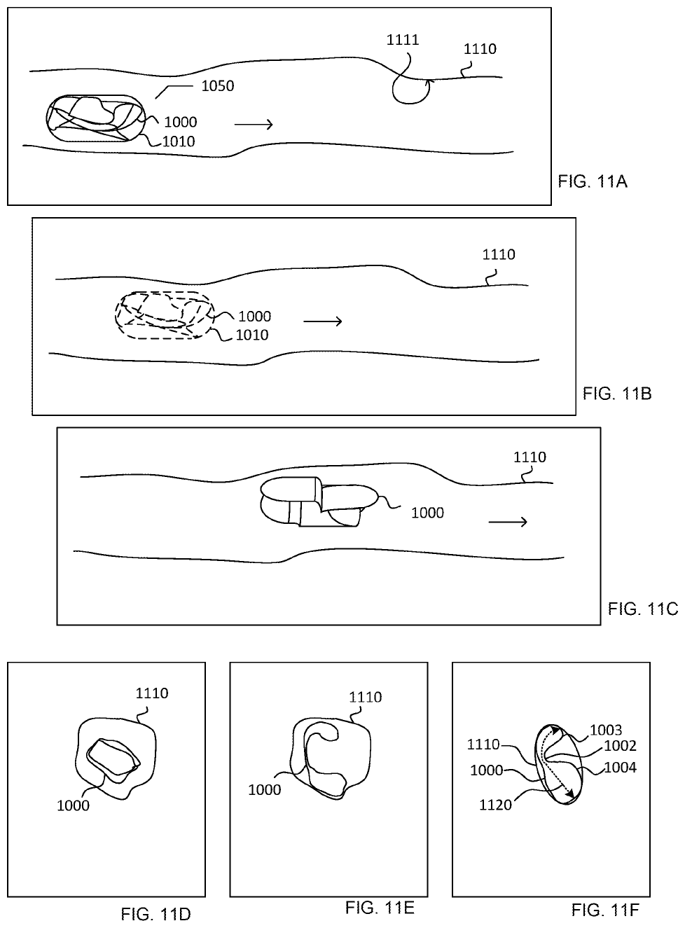

FIGS. 11A-11F illustrate an example of how device 1050 may be provided to a target delivery site in the GI tract and position expandable component 1000 to deliver a therapeutic formulation into a lumen wall 1111 of a GI lumen 1110. Referring back to FIGS. 11A-11C, device 1050 is shown from a view approximately perpendicular to a direction of travel of device 1050 (indicated by an arrow), whereas device 1050 in FIGS. 11D-11F is shown as if looking into lumen 1110 (e.g., approximately 90 degrees from the view of FIGS. 11A-11C).

FIG. 11A illustrates device 1050 within lumen 1110 shown in a relaxed state (e.g., between peristaltic contractions). In this embodiment, an outer diameter of device 1050 is less than an inner diameter of lumen 1110 at this point in the GI tract. Device 1050 traverses lumen 1110, for example through operation of peristalsis, fluid dynamics, and/or gravity.

FIG. 11B illustrates that, after reaching the designed target site or after encountering conditions (e.g., pH) representative of the designed target site, capsule 1010 degrades (or is triggered to break open or degrade) as device 1050 continues to traverse lumen 1110. For example with respect to delivery within the intestine, a target site may be in a general region (e.g., small intestine), a more specific region (e.g., jejunum), or a specific tissue area (e.g., a tissue area that has been marked or has a known or sensed condition to be treated).

FIG. 11C illustrates that, subsequent to (or concurrent with) capsule 1010 degrading, expandable component 1000 begins to unfurl from its folded and/or rolled arrangement.

FIG. 11D illustrates expandable component 1000 mostly unfurled, and FIG. 11E illustrates expandable component 1110 as it is expanded (e.g., by an expansion module).

FIG. 1 IF illustrates expandable component 1000 after expansion. In this embodiment, expandable component 1000 would have been able to expand if not constrained by lumen 1110 until a reference line 1120 was approximately a straight line. However, lumen 1110 exerts force against expandable component 1000 which causes expandable component 1000 to bend at a hinge 1002. An NCS 1003 and an NCS 1004 of expandable component 1000 then press against the lumen wall of lumen 1110 (e.g., at opposing sides of the lumen) and position expandable component 1000 to deliver a therapeutic formulation at or into the lumen wall. Hinge 1002 may also press against the lumen wall. In an embodiment, peristalsis may move expandable component 1000 in its expanded state through lumen 1110. In an embodiment, expandable component 1000 may have sufficient internal pressure (combined with surface tension) to firmly hold expandable component 1000 in approximately a same position even during peristalsis. In an embodiment, expandable component 1000 includes a surface roughness or surface protrusions to assist in maintaining a position of expandable component 1000.

Expansion of an expandable component may be accomplished by any suitable expansion module. In an embodiment, a spring mechanism is released from a compressed state to deploy a filler piece of firm or soft material to push outwards and thus expand the expandable component. In an embodiment, two or more reactants are mixed together to form a gas and thus expand the expandable component. In an embodiment, a material is rapidly combusted to generate a gas and thus expand the expandable component. Other expansion modules are within the scope of the present disclosure.

FIG. 12 illustrates a lumen 1200 in which a self-sizing device 1210 including an expandable component 1250 is positioned. In this embodiment, the force of lumen 1200 against expandable component 1250 is not sufficient to substantially cause a hinge 1251 of expandable component 1250 to bend, so expandable component 1250 is in an approximately fully-extended state. Said another way, a bending of hinge 1251 of expandable component 1250 is negligible at this position in this lumen 1200.

Expandable component 1250 is expanded by generation of gas, and includes a valve 1255 to release the gas from within expandable component 1250 into lumen 1200 to deflate expandable component 1250. Valve 1255 may include one or more components. In an embodiment, valve 1255 is a pinch valve that degrades in the presence of fluid (e.g., biological matter) to reveal a port or an opening defined by expandable component 1250. In an embodiment, valve 1255 is a piece of material or a coating over a port or an opening in expandable component 1250, and the piece of material or coating is designed to degrade a period of time after expandable component 1250 delivers a therapeutic formulation. In an embodiment, valve 1255 is opened manually.

The therapeutic formulation (or multiple therapeutic formulations) can be stored within expandable component 1250 in any constitution (e.g., as a solid, fluid, slurry, or powder). The therapeutic formulation(s) can be delivered in any constitution (e.g., as a solid, fluid, slurry, or powder). In an embodiment, two or more components of the therapeutic formulation are mixed within expandable component 1250 prior to delivery. In an embodiment, expandable component 1250 includes a portal such as portal 1260 defining an opening 1261 through which the therapeutic formulation is delivered.

In an embodiment, the therapeutic formulation is passively released such that the therapeutic formulation is applied to an inner surface of lumen 1200 (e.g., for absorption through mucosa); in such an embodiment, the therapeutic formulation may be released through a portal such as portal 1260, or may be released through multiple holes (not shown) in expandable component 1250 which are uncovered by the unfurling of expandable component 1250, are created during the unfurling, or are uncovered by degradation of a coating over the holes.

In an embodiment, the therapeutic formulation is actively released such that the therapeutic formulation is forcibly expelled from expandable component 1250 and through one or more layers of lumen 1200 or beyond (e.g., into or through the mucosa, through the submucosa, through the muscularis, through the serosa, or through the an outer wall of lumen 1200, or through the peritoneal wall into the mesentery or peritoneal cavity); in such an embodiment, the therapeutic formulation may be released through one or more portals such as portal 1260 or through one or more holes (not shown) in expandable component 1250 which are uncovered by the unfurling of expandable component 1250, are created during the unfurling, or are uncovered by degradation of a coating over the holes.

The therapeutic formulation may be forcibly expelled from expandable component 1250 by a delivery mechanism. For example, with respect to a solid composition (e.g., tablet, pellet, or pointed form), the therapeutic formulation may be expelled by way of a spring mechanism that is released to quickly eject the solid composition out of expandable component 1250, or by way of a piston mechanism in which the piston is moved by a spring mechanism or a gas expansion to push the solid composition quickly out of expandable component 1250. For example with respect to a fluid, slurry, or powder of therapeutic formulation, a bladder may be squeezed to force the fluid, slurry, or powder out of expandable component 1250.

In an embodiment, a surface of expandable component 1250 through which the therapeutic formulation is delivered contacts the wall of lumen 1200 along a stretch of approximately 5 mm to 20 mm.

In an embodiment, the therapeutic formulation is delivered from expandable component 1250 from, or along, multiple surfaces.

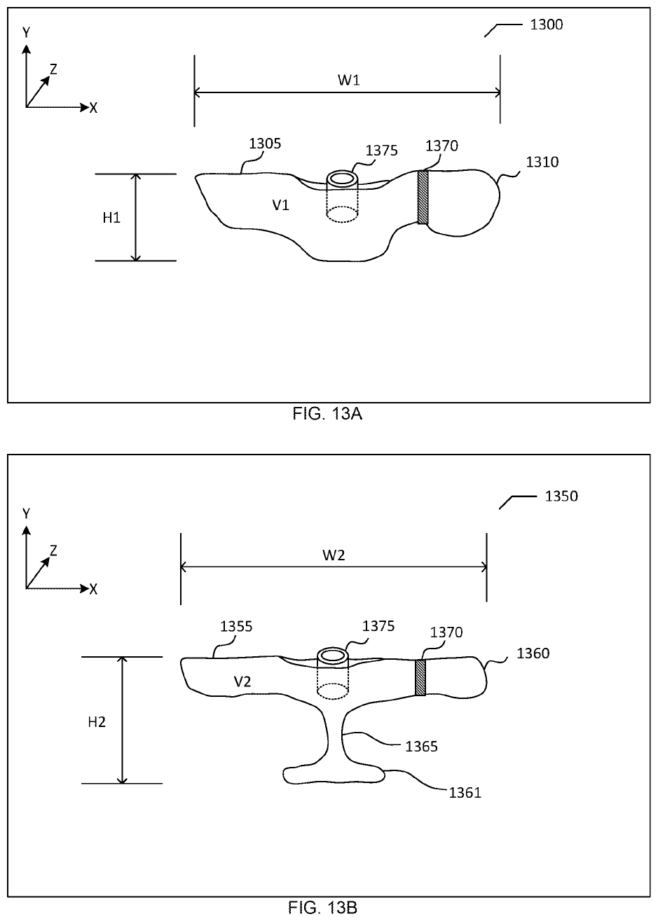

FIG. 13A illustrates an embodiment of a self-sizing device 1300 including an expandable component 1305 which includes one NCS 1310 and no hinge. FIG. 13B illustrates an embodiment of a self-sizing device 1350 including an expandable component 1355 which includes two NCSs, an NCS 1360 and an NCS 1361, and a hinge 1365 between. With respect to these embodiments, a width W1 of expandable component 1305 is similar to a width W2 of expandable component 1355, whereas a height H1 of expandable component 1305 is less than a height H2 of expandable component 1355.

Multiple devices 1300 (FIG. 13A) structured according to a same design will have approximately a same maximum circumference of NCS 1310 (in a y-z plane) when fully inflated in a lumen due to the non-conformance of NCS 1310, such that expandable component 1305 does not significantly adapt to a variety of different internal lumen circumferences. Different sizes of device 1300 may therefore be appropriate for different animalia species and/or different subjects within a species. In contrast, multiple devices 1350 (FIG. 13B) structured according to a same design will likely not have a same maximum circumference (in a y-z plane) when fully inflated in a lumen because each device 1350 will adjust to the size of the lumen in which it is inflated due to the flexibility (non-conformance, bending) of hinge 1365 even when inflated. Maximum circumference with respect to device 1350 within the lumen refers to a maximum circumference of the shape that device 1350 takes within the lumen (e.g., a partially-extended shape or a fully-extended shape).

Trial device designs similar to the illustrations of devices 1300, 1350 were prepared, and multiple trial devices were manufactured based on each of the trial device designs. For convenience of reference, the identifying numerals used in FIG. 13A and FIG. 13B are used to describe these trial devices in the following discussion. For these trial devices, an expansion module included two reactants (sodium bicarbonate and citric acid) and a biodegradable pinch valve 1370 that separated the two reactants. Valve 1370 was designed to degrade and allow the reactants to mix to generate carbon dioxide to inflate expandable component 1305 and expandable component 1355.

For the trial devices, all of the respective expandable components were constructed with similar materials, which did not significantly stretch during inflation.

Human clinical trials were performed to compare the percent delivery rate for device 1300 and device 1350 in delivering a solid composition formulation into human GI lumen walls.

Three trial device designs were used, Device A, Device B, and Device C. Device A and Device B were similar to device 1300 (FIG. 13A), and Device C was similar to device 1350 (FIG. 13B). Each device in the trials included a capsule which was swallowed by the subject (not illustrated in FIGS. 13A, 13B).

Device A had a maximum circumference (in a y-z plane) of approximately 65 mm when fully inflated and unconstrained. Device B had a maximum circumference (in a y-z plane) of approximately 68 mm when fully inflated and unconstrained. For clarity, Device A and Device B had a similar shape and similar width (a width of Device A and a width of Device B were approximately equal to W1), but Device B was taller than Device A (e.g., a height of Device A was equal to H1 and a height of Device B was greater than H1). Device C had a maximum circumference (in a y-z plane) of approximately 80 mm when fully inflated and unconstrained.

As expanded, an interior volume V1 of expandable component 1305 of Device A was approximately 8 cubic centimeters (cc), whereas an interior volume V2 of expandable component 1355 of Device C was approximately 4 cc. The reduced volume of expandable component 1355 was due in part to NCS 1360 and NCS 1361 of device 1350 each and collectively having a smaller circumference (in a y-z plane) than NCS 1310 of device 1300. The reduced volume of expandable component 1355 of Device C was achieved even though the overall height H2 of expandable component 1355 was greater than the overall height H1 of expandable component 1305 of Device A. The greater height of expandable component 1355 allowed for deployment in larger-diameter lumens. The smaller volume of expandable component 1355 allowed for a reduction in an amount of reactants needed as compared to an amount of reactants needed in expandable component 1305 to achieve a same internal pressure.

In the trial devices, a delivery mechanism 1375 included a piston that delivered a therapeutic formulation by pressure of the carbon dioxide against the piston, causing the piston to rapidly move and eject a solid composition of the formulation into a wall of the lumen.

In the trial devices, radiopaque markers were included in the therapeutic formulation and elsewhere within the trial devices. Imaging was performed throughout each trial to track a location of the respective trial device, track whether the respective expandable component was separated from the capsule, and track whether the therapeutic formulation was delivered into a lumen wall in the small intestine.

Results. Device A delivered the therapeutic formulation into the GI lumen wall in 25% of 12 subjects; Device B delivered the therapeutic formulation into the GI lumen wall in 50% of 20 subjects; and Device C delivered the therapeutic formulation into the GI lumen wall in 80% of 20 subjects. A 25%-50% delivery rate (e.g., as for Device A and Device B) would be acceptable, but an 80% delivery rate (Device C) or greater is of course preferable.

The trial results showed that increasing a circumference of the expandable component (Device A compared to Device B) resulted in a better delivery rate across a set of subjects. However, the trial results suggested that a delivery rate in a particular human may be affected by a maximum circumference (in the y-z plane) of a device with no hinge, such that, for some therapeutic treatments, each human might be tested with multiple devices (each with different maximum circumference) prior to or at the beginning of the therapeutic treatment, to identify which device size was the most appropriate to use for that human.

In contrast, the trial results suggested that a single size and design of an expandable component including a hinge (e.g., Device C) could be used successfully across a high percentage of subjects. Notably, the results achieved by Device C were achieved using an initial version of a hinged design; it is expected that improvements to the hinged design will result in an even higher delivery rate.

Further of note is that multiple lots of self-sizing devices structured similarly to device 1350 with expandable component 1355 having hinge 1365 (e.g., similar to Device C), all having the same design and dimensions of expandable component 1355, were tested in various animalia species including humans, pigs, and dogs, with the result being high delivery rates in each species, even though the intestinal lumen sizes of the various species differed significantly; by comparison, for devices structured similarly to device 1300 with expandable component 1305 having no hinge, multiple sizes of expandable component 1305 were used across different animalia species to adapt to lumen size of each particular species to avoid discomfort caused by the intestines being excessively stretched.

For devices structured similarly to Device C, it was shown that delivery rate was consistent whether the subjects were fed, or were fasted for several hours prior to ingestion of the device.

Prior to the conception of the hinge structure for an expandable component, concerns included how many device sizes should be made available and how to determine an appropriate device size for an individual. The clinical trial results show that the novel concept of adding a hinge to an expandable component solves these and other concerns. A single design of a hinged expandable component in accordance with the present disclosure can deliver a formulation into a lumen with a high rate of delivery over a selected range of circumferences of an inner wall of the lumen. In an embodiment, a device is designed for delivery within the small intestine in humans, and the selected range is about 10 mm to about 100 mm, or about 20 mm to about 80 mm. In an embodiment, a self-sizing device suitable for use at any of various locations along the GI tract (e.g., stomach, small intestine, large intestine, colon) over a variety of animalia species is designed to have a selected range of about 5 mm to about 500 mm. Other ranges may be selected, and different ranges may be selected for different types of lumens within the body, or for other types of lumens.

In an embodiment, an expandable component is designed with at least one hinge visible from a first viewing angle and multiple hinges visible from a second viewing angle rotated from the first viewing angle, such that each of three or more sections (NCS and/or hinge sections) of the expandable component contacts a lumen in which the expandable component is disposed. This structure may allow compliance of multiple hinges to adapt the expandable component to a wider selected range of lumen circumferences.

Another benefit of the design concept using a hinge is that a total surface area of the expandable component may be reduced as compared to an expandable component not using a hinge, while still allowing for a single self-sizing device design to be used for applications in a wide variety of lumens, in a wide variety of animalia species, and in a wide variety of subjects within a species. Accordingly, a reduced amount of material may be needed; this reduction in material, along with a reduced amount of reactants, may result in cost savings.

In an embodiment, an expandable component is structured using one type of material throughout. In an embodiment, an expandable component is structured using two or more different materials. In an embodiment, a level of compliance of an expandable component may be adjusted based on a thickness of material at certain areas of the expandable component. In an embodiment, a level of compliance of an expandable component may be adjusted by adding layers of a same material or different materials at certain areas of the expandable component. Examples of materials which may be used to structure an expandable component include polyester, PET, HDPE, a cross linked polymer, silicone, polyurethane, or other elastomer or polymer.

In an embodiment, a formulation is in a solid composition. A delivery mechanism (e.g., delivery mechanism 1375) includes an advancement device (e.g., a piston or rod) directly or otherwise operably coupled to the solid composition to exert a force on a surface of the solid composition to advance it into a lumen wall. The delivery mechanism may be in operative contact with or otherwise closely positioned adjacent to the lumen wall when the expandable component is expanded, such that the solid composition is ejected directly into the lumen wall with a minimal gap or no gap between a surface of the delivery mechanism and the lumen wall, which may improve a delivery rate of the self-sizing device.

In an embodiment, a self-sizing device includes a solid composition detachably coupled to a piston so that after advancement of the solid composition to the lumen wall, the solid composition detaches from the piston.

Specific materials can be chosen to confer desired structural and material properties to the solid composition (e.g., column strength for insertion into the lumen wall, or porosity and/or hydrophilicity for controlling disintegration of the solid composition and thus the release of a therapeutic formulation). In an embodiment, a tip of the solid composition includes or is coated with a degradable material such as sucrose, maltose, or other sugar, to increase hardness and tissue piercing properties of the tip. Once positioned within a lumen wall, the solid composition may be degraded by interstitial fluids within tissue so that the therapeutic formulation dissolves and is absorbed into the blood stream. Properties of a solid composition such as size, shape, and chemical composition can be selected to allow for dissolution and absorption of a drug in a matter of seconds, minutes, or hours. Rates of dissolution can be controlled through various excipients such as disintegrants (e.g., starch, sodium starch glycolate, or a cross-linked polymer such as carboxymethyl cellulose). The choice of disintegrants may be specifically adjusted for the environment within a lumen wall (e.g., blood flow and average number of peristaltic contractions).

A solid composition can be fabricated entirely from a formulation, or can define a cavity that includes a formulation. A self-sizing device can include and deliver one or more solid compositions, each of which can contain the same or a different formulation from another of the solid compositions. Each solid composition can have different properties; for example, two solid compositions may be designed to deliver respective formulations concurrently (e.g., two instances of one formulation, or one instance each of two formulations), or may be designed to deliver respective formulations at different times (e.g., to provide a subsequent dose of a same formulation or to provide a different formulation subsequently).

While the present disclosure has been described and illustrated with reference to specific embodiments thereof, these descriptions and illustrations do not limit the present disclosure. It can be clearly understood that various changes can be made, and equivalent components can be substituted within the embodiments, without departing from the true spirit and scope of the present disclosure as defined by the appended claims. Also, components, characteristics, or acts from one embodiment can be readily recombined or substituted with one or more components, characteristics or acts from other embodiments to structure numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit, and scope of the present disclosure. All such modifications are intended to be within the scope of the claims appended hereto. While the methods disclosed herein have been described with reference to particular operations performed in a particular order, it can be understood that these operations can be combined, sub-divided, or re-ordered to an equivalent method without departing from the teachings of the present disclosure. Therefore, unless specifically indicated herein, the order and grouping of the operations are not limitations of the present disclosure.

What is claimed is:

1. A self-sizing device for delivering a therapeutic formulation, the device comprising:
a capsule sized and structured to be orally ingested; and
an expandable component disposed within the capsule, the expandable component including at least one non-compliant section structured to resist deformation in an expanded configuration, the expandable component further including at least one hinge structured to allow deformation in an expanded configuration, the expandable component structured to expand within a lumen and bend about the hinge to adapt to a circumference of an inner wall of the lumen independent of the circumference of the inner wall within a selected range.

2. The device of claim 1, wherein the selected range is about 50 mm to about 150 mm.

3. The device of claim 1, wherein, while in the capsule, the expandable component is in a folded and/or rolled arrangement and is structured to unfold and/or unroll when released from the capsule to expand to a maximum circumference in the absence of a constraining force and to less than a maximum circumference in the presence of a constraining force.

4. The device of claim 1, wherein the hinge has a width or a circumference smaller than a respective width or circumference of the non-compliant section.

5. The device of claim 1, wherein the lumen is a lumen of the small intestine.

6. The device of claim 1, wherein the expandable component comprises at least two non-compliant sections.

7. The device of claim 1, wherein the expandable component comprises at least two hinges.

8. The device of claim 1, further comprising the therapeutic formulation and a piston, wherein the self-sizing device is structured to, upon expansion of the expandable component, cause the piston to apply a force to the therapeutic formulation, the force designed to expel the therapeutic formulation from the device.

9. A system for delivering a formulation, the system comprising:
a self-sizing device comprising an expandable component that includes at least one non-compliant section structured to resist deformation in an expanded configuration, the expandable component further including at least one hinge structured to allow deformation in an expanded configuration, the expandable component structured to bend about the hinge upon expansion to adapt to an inner circumference of a lumen independent of the inner circumference of the lumen within a selected range;
the formulation; and
a delivery mechanism, wherein the self-sizing device is structured to, upon expansion of the expandable component, cause the delivery mechanism to apply a force to the formulation, the force designed to expel the formulation from the self-sizing device.

10. The system of claim 9, further comprising a capsule in which the expandable component is disposed, wherein, while in the capsule, the expandable component is in a folded and/or rolled arrangement and is structured to unfold and/or unroll when released from the capsule.

11. The system of claim 10, wherein the expandable component is further structured to, after being released from the capsule, expand to a maximum circumference in the absence of a constraining force and expand to less than a maximum circumference in the presence of a constraining force.

12. The system of claim 9, wherein the self-sizing device is structured to be disposed in a gastrointestinal tract, and the selected range is about 50 mm to about 150 mm.

13. The system of claim 9, wherein the hinge has a width or a circumference smaller than a respective width or circumference of the non-compliant section.

14. The system of claim 9, wherein the lumen is a lumen of the small intestine.

15. The system of claim 9, wherein the expandable component comprises at least two non-compliant sections.

16. The system of claim 9, wherein the expandable component comprises at least two hinges.

17. The system of claim 9, wherein the delivery mechanism comprises a piston, and the self-sizing device is structured such that upon expansion of the expandable component the piston applies the force to the formulation.

18. A method for delivering a therapeutic formulation, comprising:
providing a self-sizing device comprising the therapeutic formulation, the self-sizing device further comprising an expandable component that includes at least one non-compliant section structured to resist deformation in an expanded configuration, the expandable component further including at least one hinge structured to allow deformation in an expanded configuration, the expandable component structured to bend about the hinge upon expansion to adapt to an inner circumference of a lumen independent of the inner circumference of the lumen within a selected range; and
providing instructions for disposing the self-sizing device within a body to deliver the therapeutic formulation into the lumen.

19. The method of claim 18, wherein the instructions include instructions for swallowing the self-sizing device.

20. The method of claim 18, wherein the instructions include instructions for manually inserting the self-sizing device into a lumen of a body.
